(19) United States
(12) Patent Application Publication
(10) Pub. No.: US 2023/0109943 A1
(43) Pub. Date: Apr. 13, 2023

(54) MULTI-DIRECTIONAL DEFLECTABLE CATHETER WITH SELECTIVE STIFFENING

(71) Applicant: THE REGENTS OF THE UNIVERSITY OF CALIFORNIA, Oakland, CA (US)

(72) Inventors: Peter Hsiue, Oakland, CA (US); Marcus Goudie, Oakland, CA (US); Lucas Allera, Oakland, CA (US); Renea Sturm, Oakland, CA (US)

(21) Appl. No.: 17/906,361

(22) PCT Filed: Mar. 17, 2021
(86) PCT No.: PCT/US21/22720

§ 371 Date: Sep. 15, 2022

Related U.S. Application Data
(60) Provisional application No. 63/118,563, filed on Nov. 25, 2020.
(60) Provisional application No. 62/990,728, filed on Mar. 17, 2020.

(51) Int. Cl. A61M 25/01

(52) U.S. Cl. CPC A61M 25/0155; A61M 25/0138

(57) ABSTRACT

The present invention provides a variable stiffness catheter comprising a pneumatic actuator lumen system that allows manipulation of shape and curvature at one or more locations along the catheter.

### CROSS-REFERENCE TO RELATED APPLICATIONS

This application claims priority to U.S. Provisional Patent Application No. 62/990,728, filed Mar. 17, 2020, and to U.S. Provisional Patent Application No. 63/118,563, filed Nov. 25, 2020, the contents of which are each incorporated by reference herein in their entirety.

### BACKGROUND OF THE INVENTION

Neurointerventional radiology (NIR) is a rapidly growing procedural subspecialty that leverages advanced radiologic imaging and minimally invasive endovascular techniques to treat threatening conditions of the central nervous system. Regardless of the pathology, nearly all NIR procedures rely on the careful navigation of catheters through the complex cerebral vasculature to reach a specific treatment area. Difficulty with or the inability to successfully navigate a segment of vasculature can result in increased procedure time and cost (exchanging several different catheters and guidewires); increased risk of intraprocedural complications (fragile vessels may perforate with repeated pass attempts); and finally decreased specificity of treatment (release of treatment agent more proximally than intended). Traditionally, NIR procedures are carried out using a lower extremity arterial access point at the femoral artery. Utilization of this transfemoral access (TFA) is still the most common approach, but there has been growing interest in other arterial access points such as the radial artery or transradial access (TRA). Popularized by interventional cardiologists, TRA has been shown to reduce bleeding complications, reduce all-cause mortality, expedite patient recovery, improve patient satisfaction, and reduce healthcare costs when compared to TFA in the interventional cardiology literature. However, adoption of TRA by NIR has been slow due to a variety of anatomic challenges presented in the approach that increase the difficulty of device navigation. Specifically, selective access of the supra aortic arterial branches that lead to the cerebral vasculature can be particularly difficult due to acute angle branching and vessel tortuosity. Furthermore, unlike in interventional cardiology, there are no TRA-specific devices currently available which means that NIR proceduralists need to rely on TFA devices for an entirely different vascular access point. Thus, there is a need in the art for an improved catheter system with a manipulatable shape and curvature capable of navigating tight turns in anatomy, such as transradial access (TRA) procedures for neuro-interventional radiology. This technology has the potential to not only improve NIR clinical outcomes by increasing the speed and specificity of procedures, but also reduce healthcare resource utilization by decreasing the number of devices needed for a procedure. The present invention meets this need.

### SUMMARY OF THE INVENTION

In one aspect, the present invention relates to a variable stiffness catheter comprising: a flexible elongated tube having a tube wall surrounding a central lumen, the tube extending by a length between an open proximal end and an open distal end and comprising a series of alternating flexible and stiff sections; and a plurality of actuator lumens embedded within the tube wall, each actuator lumen having a proximal inlet and being fluidly connected to at least one inflatable actuator segment disposed within the tube wall, wherein each actuator segment spans at least one flexible section. In one embodiment, the at least one actuator segment is arranged radially around the central lumen. In one embodiment, the length of the tube wall is divided into sections, such that each section comprises at least one actuator segment. In one embodiment, the catheter further comprises a fluid source configured to be in fluid communication with the proximal inlet of each actuator lumen. In one embodiment, each actuator segment is configured to preferentially expand when the plurality of fluidly connected chambers is pressurized by a fluid, causing the tube wall to bend. In one embodiment, expansion of each actuator segment does not alter an outer diameter of the catheter. In one embodiment, the bend is between 0 degrees and 180 degrees relative to a longitudinal axis of the flexible elongated tube. In one embodiment, the fluid pressurizes the fluidly connected chambers at a pressure of between about 0 and 500 kPa. In one embodiment, each actuator segment is configured to contract when the plurality of fluidly connected chambers is exposed to a vacuum, causing the tube wall to stiffen.

In one embodiment, the plurality of actuator lumens are symmetrically placed around the elongated tube. In one embodiment, the plurality of actuator lumens are asymmetrically placed around the elongated tube. In one embodiment, the flexible sections have a Shore hardness between about 10A and 40A. In one embodiment, the stiff sections have a Shore hardness between about 40A and 70A. In one embodiment, the flexible elongated tube comprises an outer diameter between about 1 mm and 50 mm. In one embodiment, the central lumen comprises an inner diameter between about 0.5 mm and 49.5 mm. In one embodiment, the length is between about 5 mm and 5 m.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1 depicts a schematic of an exemplary variable stiffness catheter of the present invention.

FIG. 2 depicts a cross-sectional view of an exemplary variable stiffness catheter of the present invention.

FIG. 3 depicts a partial side cross-sectional view of an exemplary variable stiffness catheter of the present invention.

FIG. 4 depicts deformation of an exemplary variable stiffness catheter of the present invention. (Left) Inflating an actuator segment with increasing pressure is shown not to change an outer diameter of the catheter. (Right) Inflating an actuator segment with increasing pressure increases a bending angle of the catheter.

FIG. 5 depicts images of molds for fabricating a section of a variable stiffness catheter wall in two different scales (top: 5X; bottom: 10X).

FIG. 6 depicts images of sections of a variable stiffness catheter wall fabricated from the molds depicted in FIG. 5.

### DETAILED DESCRIPTION

The present invention provides an improved catheter systems comprising a pneumatic system that allows manipulation of shape and curvature at one or more locations along the catheter for use in neuro-interventional radiology.

### Definitions

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

Description

The present invention centers around the ability to manipulate the shape, curvature, and support of a catheter delivery system focused on (but not limited to) use for transradial access (TRA) procedures for neuro-interventional radiology. The catheter system utilizes principles from soft robotics where a pneumatic system is used to modulate the shape and structure of the catheter at one or more locations along the catheter length. These pneumatic actuators are embedded within the wall of the catheter, which act as a hollow structure where fluids can be applied to asymmetrically apply force to the catheter wall, inducing a bend. The asymmetric force applied to the wall comes through applying pressure. However, the structure of the actuator also allows for an increase in the stiffness of the catheter by application of a vacuum. Fluids to the actuators are delivered through a lumen along the catheter within a medial layer between the outer wall and central lumen where medical devices can be deployed.

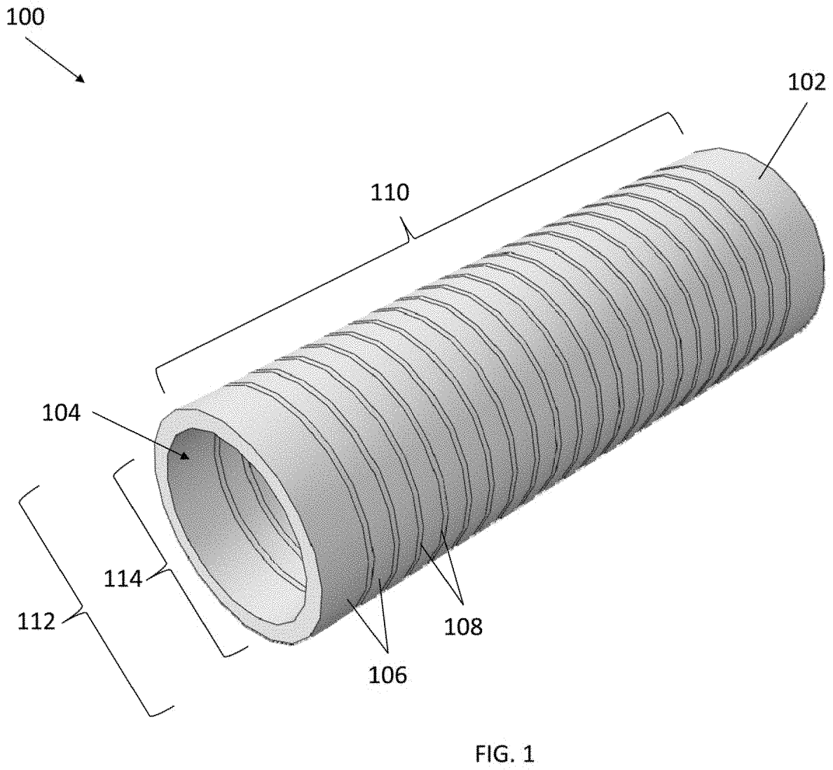

Referring now to FIG. 1, an exemplary variable stiffness catheter 100 is depicted. Catheter 100 comprises an elongate tubular catheter wall 102 having a lumen 104 running therethrough. Catheter 100 comprises a length 110, an outer diameter 112, and an inner diameter 114. Each of the dimensions of catheter 100 can have any desired size. For example, length 110 can be between about 5 mm and about 5 m. Outer diameter 112 can have a diameter between about 1 mm to about 50 mm. Inner diameter 114 can have a diameter between about 0.5 mm to about 49.5 mm. Catheter 100 comprises a series of alternating rings of flexible sections 106 and stiff sections 108. Flexible sections 106 enable catheter 100 to bend, while stiff sections 108 are rigid and have reduced bending or prevent bending. While FIG. 1 depicts flexible sections 106 as rings that are longer than stiff sections 108, it should be understood that flexible sections 106 and stiff sections 108 can have any desired length, such as a length between about 1 mm and about 100 mm or greater. It should also be understood that flexible sections 106 and stiff sections 108 can each comprise different length at different locations along length 110 of catheter 100. For example, a proximal section of catheter 100 can comprise an uninterrupted length of a stiff section 108 to facilitate insertion of instruments into lumen 104 and to serve as a handle; a section of catheter 100 configured to form a tight bend over a short length can comprise a series of alternating flexible sections 106 and stiff sections 108, each comprising a short length; and a section of catheter 100 configured to form a gradual bend over a long length can comprise a series of alternating flexible sections 106 and stiff sections 108, each comprising a long length. In various embodiments, flexible sections 106 and stiff sections 108 can be described in terms of Shore hardness. For example, flexible sections 106 can have a Shore hardness between about 10A and 40A, and stiff sections 108 can have a Shore hardness between about 40A and 70A or greater. In some embodiments, flexible sections 106 and stiff sections 108 can each be formed from different materials, wherein the differing materials impart differing Shore hardness to each section. In some embodiments, flexible sections 106 and stiff sections 108 can each be formed from the same material, wherein differing Shore hardness in each section is derived from differing material thickness, as will be described elsewhere herein.

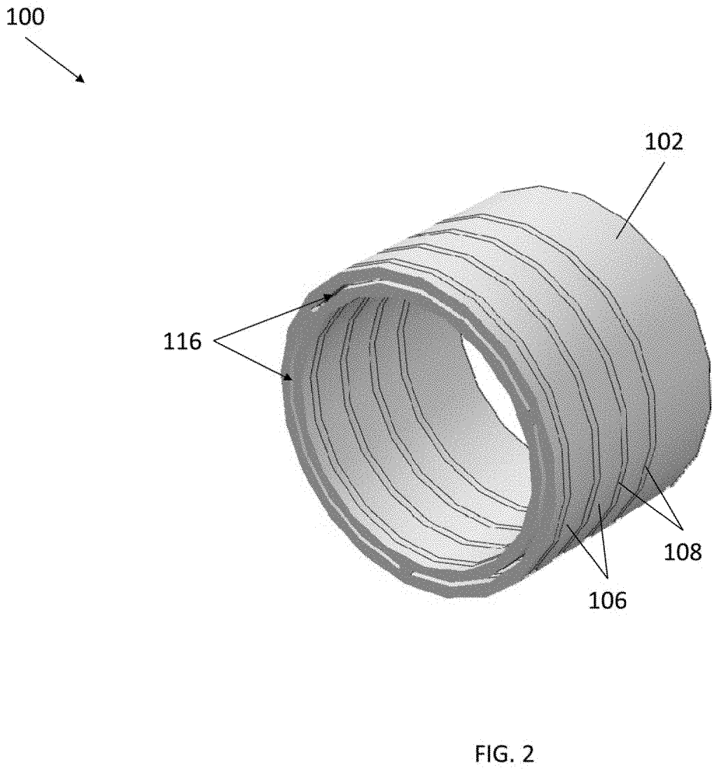

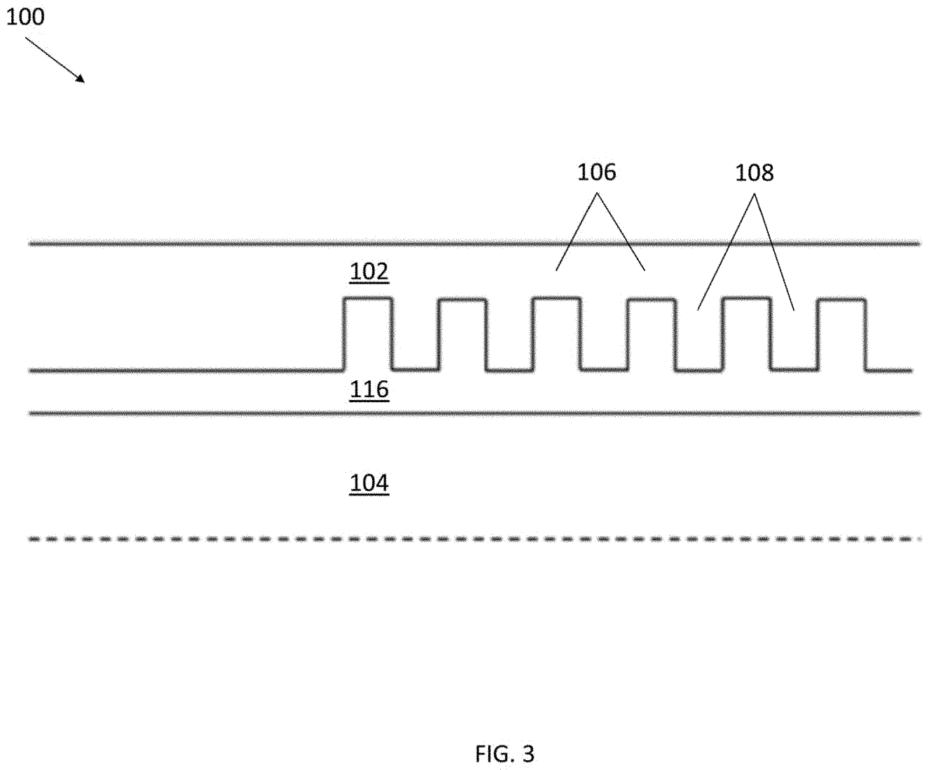

Referring now to FIG. 2, a cross-sectional view of a section of catheter 100 is depicted. Catheter 100 comprises one or more actuator segment 116 positioned within wall 102, wherein each actuator segment 116 spans at least one flexible section 106 and can span a plurality of flexible sections 106 and stiff sections 108 along length 110 of catheter 100. One example is shown in the cross-sectional view of FIG. 3, where flexible sections 106 are shown being constructed from a thinner material relative to stiff sections 108 being constructed from a thicker material. Each actuator segment 116 is fluidly connected to a proximal inlet by an actuator lumen, wherein the proximal inlet can be engaged to an actuator controller (lumen shown branching in a leftward direction in FIG. 3; actuator controller not shown). In some embodiments, actuator segment 116 is inflatable by a fluid, such as a gas, a liquid, or a gel, wherein inflation by the fluid expands actuator segment 116 to induce a curve in a portion of catheter 100 in which the actuator segment 116 resides. The amount of inflation can be described in terms of pressure, such as a pressure between about 0 kPa and 500 kPa or greater. In some embodiments, actuator segment 116 is deflatable by a vacuum source, wherein deflation contracts actuator segment 116 to stiffen a portion of catheter 100 in which the actuator segment 116 resides. Accordingly, the actuator controller to which each actuator segment 116 is fluidly connected can be a fluid source, a vacuum source, or both.

While FIG. 2 depicts three actuator segments 116 of equal size radially positioned equidistantly within wall 102, it should be understood that catheter 100 can comprise any desired number of actuator segments 116 in any desired pattern. In various embodiments, a radial section of catheter 100 can comprise between about 1 and 10 or more actuator segments 116, and catheter 100 can comprise one or more discrete sections having separately controllable actuator segments 116. For example, a section of catheter 100 can comprise a single actuator segment 116 positioned in wall 102, wherein the single actuator segment 116 is configured to induce a bend in catheter 100 in a direction opposite to its position in wall 102 when inflated, and the single actuator segment 116 can span between about 10% and 90% of a radial section of wall 102. In another example, a section of catheter 100 can comprise four radial actuator segments 116 positioned in wall 102 configured to induce bends in catheter 100 as described above, wherein each actuator segment 116 can span between about 10% and 70% of a radial section of wall 102.

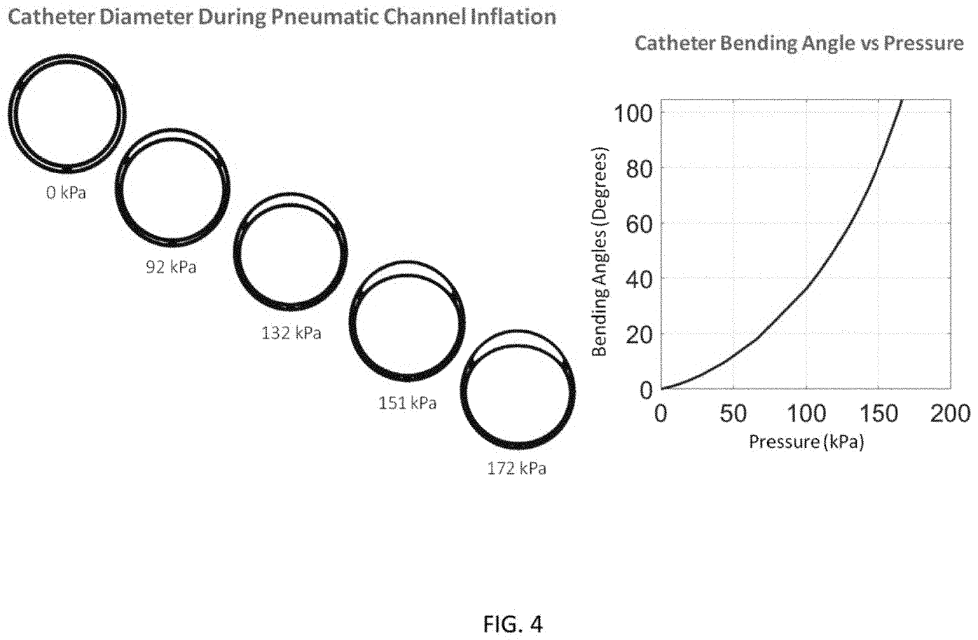

As described elsewhere herein, actuator segments 116 can be inflated with a fluid to induce a bend in catheter 100, and can also be deflated by a vacuum to stiffen catheter 100. Referring now to FIG. 4, inflation of an exemplary actuator segment 116 is shown. In the left diagram of FIG. 4, an actuator segment 116 is shown in an inflated state under progressively higher pressures. As the actuator segment 116 is inflated, it should be noted that the outer diameter of the catheter does not change. Rather, deformation can be effected internally on the inflated actuator segment 116, on adjacent actuator segments 116, longitudinally along catheter 100 (such that length increases), or combinations thereof. Inflating actuator segment 116 induces a bend in catheter 100 that increases with higher pressures, as shown in the right graph of FIG. 4. In the diagram shown in FIG. 4, inflating the top actuator segment 116 induces a bend in catheter 100 in a direction opposite to the side of the wall that the actuator segment resides, which in this example would be a downward direction. Catheter 100 can be induced to bend at an angle between about 0° and 180° or greater relative to a longitudinal axis of catheter 100. The catheters of the present invention can be made using any suitable method known in the art. The method of making may vary depending on the materials used. For example, components substantially comprising a metal may be milled from a larger block of metal or may be cast from molten metal. Likewise, components substantially comprising a plastic or polymer may be milled from a larger block, cast, or injection molded. In some embodiments, the devices may be made using 3D printing or other additive manufacturing techniques commonly used in the art.

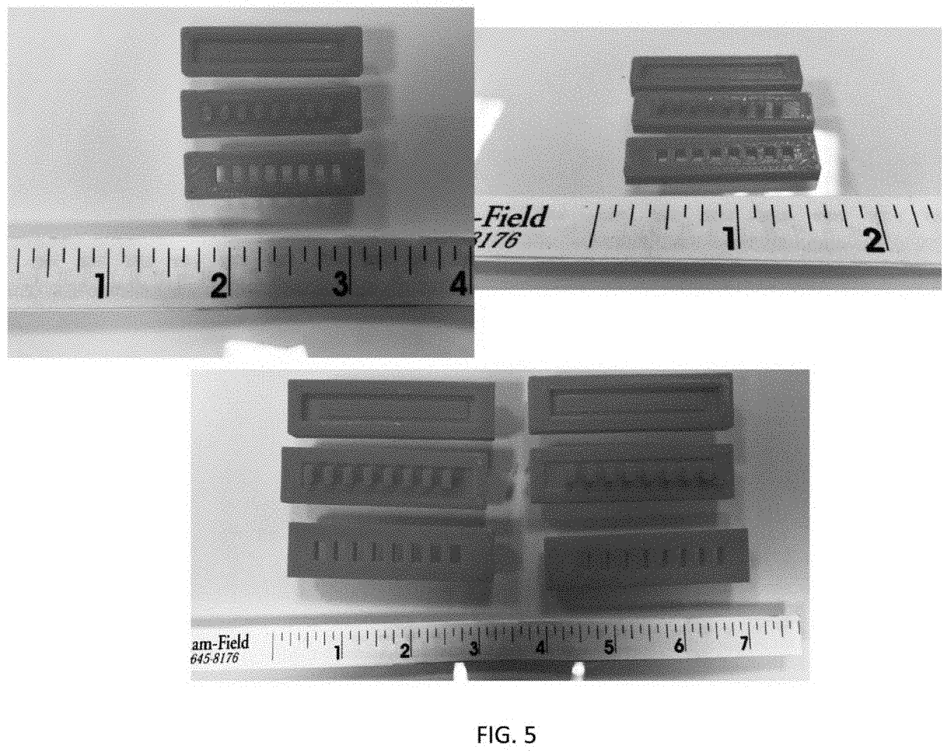

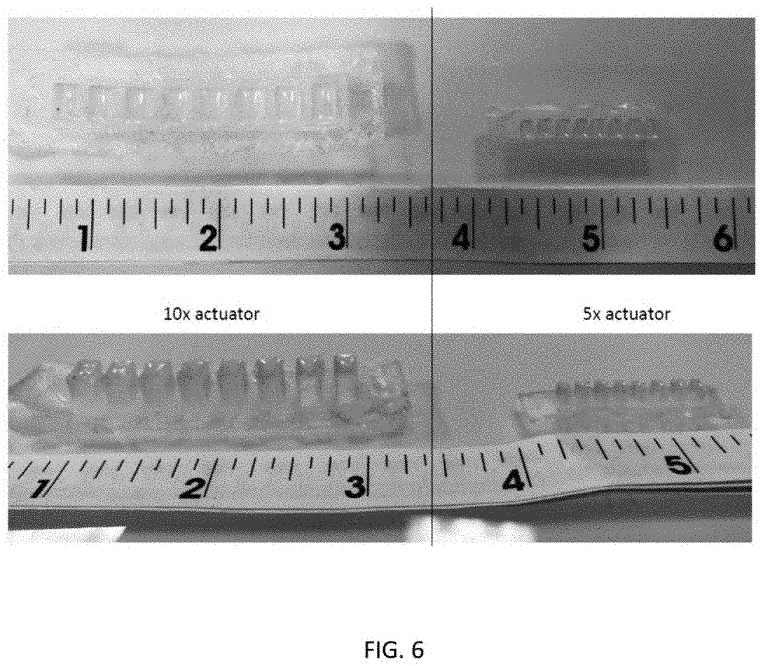

Referring now to FIG. 5 and FIG. 6, an exemplary mold is depicted, wherein the mold fabricates a section of a variable stiffness catheter wall in two different scales, a smaller scale in the upper images and a larger scale in the lower images. Each mold is shown next to a ruler for scale and is presented in three pieces: the top piece defines an outer surface of the catheter wall section, the middle piece defines an inner surface of the catheter wall section and comprises pillars that form actuator segment spaces, and the bottom piece defines the size of the actuator segments and comprises openings configured to fit around each pillar of the middle piece with a spacing that forms actuator segment walls. FIG. 6 depicts a section of catheter wall (left) fabricated using a larger scale mold and a section of catheter wall (right) fabricated using a smaller scale mold, wherein an outer surface of the catheter wall section rests on the table top and the pillars represent inflatable or deflatable actuator segments.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A variable stiffness catheter comprising:
a flexible elongated tube having a tube wall surrounding a central lumen, the tube extending by a length between an open proximal end and an open distal end and comprising a series of alternating flexible and stiff sections; and
a plurality of actuator lumens embedded within the tube wall, each actuator lumen having a proximal inlet and being fluidly connected to at least one inflatable actuator segment disposed within the tube wall, wherein each actuator segment spans at least one flexible section.

2. The variable stiffness catheter of claim 1, wherein the at least one actuator segment is arranged radially around the central lumen.

3. The variable stiffness catheter of claim 1, wherein the length of the tube wall is divided into sections, such that each section comprises at least one actuator segment.

4. The variable stiffness catheter of claim 1, further comprising a fluid source configured to be in fluid communication with the proximal inlet of each actuator lumen.

5. The variable stiffness catheter of claim 1, wherein each actuator segment is configured to preferentially expand when thea plurality of fluidly connected chambers is pressurized by a fluid, causing the tube wall to bend.

6. The variable stiffness catheter of claim 5, wherein expansion of each actuator segment does not alter an outer diameter of the catheter.

7. The variable stiffness catheter of claim 5, wherein the bend is between 0 degrees and 180 degrees relative to a longitudinal axis of the flexible elongated tube.

8. The variable stiffness catheter of claim 5, wherein the fluid pressurizes the plurality of fluidly connected chambers at a pressure of between about 0 and 500 kPa.

9. The variable stiffness catheter of claim 1, wherein each actuator segment is configured to contract when thea plurality of fluidly connected chambers is exposed to a vacuum, causing the tube wall to stiffen.

10. The variable stiffness catheter of claim 1, wherein the plurality of actuator lumens are symmetrically placed around the flexible elongated tube.

11. The variable stiffness catheter of claim 1, wherein the plurality of actuator lumens are asymmetrically placed around the flexible elongated tube.

12. The variable stiffness catheter of claim 1, wherein the flexible sections have a Shore hardness between about 10A and 40A.

13. The variable stiffness catheter of claim 1, wherein the stiff sections have a Shore hardness between about 40A and 70A.

14. The variable stiffness catheter of claim 1, wherein the flexible elongated tube comprises an outer diameter between about 1 mm and 50 mm.

15. The variable stiffness catheter of claim 1, wherein the central lumen comprises an inner diameter between about 0.5 mm and 49.5 mm.

16. The variable stiffness catheter of claim 1, wherein the length is between about 5 mm and 5 m.

17. A variable stiffness catheter comprising:
a flexible elongated tube having a tube wall surrounding a central lumen, the tube extending by a length between an open proximal end and an open distal end and comprising a series of alternating flexible and stiff sections; and
a plurality of actuator lumens embedded within the tube wall, each actuator lumen having a proximal inlet and being fluidly connected to at least one inflatable actuator segment disposed within the tube wall, wherein each actuator segment spans at least one flexible section;
wherein the proximal inlet is engaged to an actuator controller.

18. The variable stiffness catheter of claim 17, wherein the actuator controller is fluidly connected to a fluid source.

19. The variable stiffness catheter of claim 17, wherein the actuator controller is fluidly connected to a vacuum.

20. The variable stiffness catheter of claim 17, wherein the actuator segments are inflatable by a fluid to induce a curve in the catheter.
